(19) United States
(12) Patent Application Publication
(10) Pub. No.: US 2024/0327778 A1
(43) Pub. Date: Oct. 3, 2024

(54) APPARATUS AND METHODS FOR TREATING MEDIA IN LARGE-SCALE BIOREACTORS

(71) Applicant: PBS BIOTECH, INC., Camarillo, CA (US)

(72) Inventor: Chanyong Brian Lee, Newbury Park, CA (US)

(21) Appl. No.: 18/739,392

(22) Filed: Jun. 11, 2024

Related U.S. Application Data
(63) Continuation of application No. PCT/US22/53580, filed on Dec. 21, 2022.
(60) Provisional application No. 63/293,021, filed on Dec. 22, 2021.

(51) Int. Cl. C12M 1/36; C12M 1/00; C12M 1/04; C12M 1/06; C12M 1/34

(52) U.S. Cl. CPC C12M 41/48; C12M 23/24; C12M 23/28; C12M 23/34; C12M 27/02; C12M 29/08; C12M 41/26; C12M 41/34

(57) ABSTRACT

Systems and methods for treating media in large-scale bioreactors can include a method of using a large-scale bioreactor system. The method can include (a) mixing a mixture of at least cells, and media in a bioreactor, and (b) removing a portion of the media through an output port of the bioreactor. The method can also include (c) retaining the cells within the bioreactor using a screen covering the port. The method can also include (d) receiving a second media, which may in some examples include the portion of the first media removed from the bioreactor, into a media preparation container external to the bioreactor, (e) treating the second media within the media preparation container to produce oxygenated media within a threshold pH range, and (f) introducing the oxygenated media into the bioreactor using a return port of the bioreactor.

### CROSS-REFERENCE TO RELATED APPLICATIONS

This application is a Continuation of PCT/US22/53508 filed Dec. 21, 2022, which claims priority to U.S. Provisional Patent Application No. 63/293,021, filed Dec. 22, 2021, the entire contents of which are incorporated by reference herein.

### FIELD OF THE DISCLOSURE

The present disclosure relates to bioreactor systems and, more particularly, to systems and methods for treating media in bioreactor systems.

### BACKGROUND

The commercial potential of cell therapies requires overcoming the challenges related to scalable manufacturing of living cells. Therapeutic cells are typically human-derived, and as aerobic organisms they require constant management of metabolic nutrients and waste products. Furthermore, they are also typical anchorage-dependent and grow as cell aggregates or on the surface of plastic microcarriers while suspended in liquid medium. T For cell culture processes performed in bioreactors, oxygen must be dissolved into the liquid medium while dissolved carbon dioxide (CO₂) produced by the cells must be removed.

### SUMMARY

In accordance with one implementation, the present disclosure provides a method of using a large-scale bioreactor system. The method includes (a) mixing a mixture of at least cells, and a first media in a bioreactor. The method further includes (b) removing a portion of the first media through an output port of the bioreactor. The method further includes (c) retaining the cells within the bioreactor using a screen covering the output port. The method further includes (d) receiving a second media within a media preparation container external to the bioreactor. The method further includes (e) treating the second media within the media preparation container to produce oxygenated media within a threshold pH range. And the method further includes (f) introducing the oxygenated media to the bioreactor using a return port of the bioreactor.

In some implementations, the second media comprises the portion of the first media, and receiving the second media within the media preparation container comprises receiving the portion of the first media removed from the output port of the bioreactor.

In some implementations, the method further comprises moving the portion of the first media to a waste container after removing the portion of the first media from the bioreactor, and separately supplying the second media to the media preparation container.

In some implementations, treating the second media comprises adding at least one of oxygen, air, carbon dioxide, or a liquid base to the second media.

In some implementations, treating the second media comprises removing dissolved carbon dioxide from the second media.

In some implementations, the cells comprise pluripotent stem cells.

In some implementations, treating the second media comprises treating the second media using an external gas exchange device.

In some implementations, steps (b)-(f) referred to above occur substantially continuously.

In some implementations, step (a) referred to above stops while steps (b)-(f) occur.

In some implementations, steps (b)-(f) referred to above occur on demand.

In some implementations, the method further comprises warming the second media within the media preparation container.

In some implementations, treating the second media comprises sparging the second media within the media preparation container.

In some implementations, sparging is performed without the use of surfactants and without the use of anti-foaming agents.

In some implementations, the method further comprises determining an oxygen value within the mixture, comparing the determined oxygen value to a reference oxygen value, determining a difference between the determined oxygen value and the reference oxygen value being greater than a threshold value, and determining a volume of the portion of the first media to remove from the output port of the bioreactor to allow a subsequently determined oxygen value after introducing the oxygenated media to the bioreactor to be within a threshold of the reference oxygen value.

In some implementations, the method further comprises determining a parameter value within the mixture, comparing the determined parameter value to a reference parameter value, determining a difference between the determined parameter value and the reference parameter value being greater than a threshold value, and determining a volume of the portion of the first media to remove from the output port of the bioreactor to allow a subsequently determined parameter value after introducing the oxygenated media to the bioreactor to be within a threshold of the reference parameter value.

In some implementations, the parameter value comprises a value of dissolved oxygen within the mixture.

In some implementations, the parameter value comprises a value of dissolved carbon dioxide within the mixture.

In some implementations, the parameter value comprises a pH value of the mixture.

In some implementations, introducing the oxygenated media to the bioreactor using the return port of the bioreactor comprises introducing the oxygenated media to the bioreactor using the return port having a tube including a distal end that tapers toward an internal surface of the bioreactor to prevent or inhibit splashing from occurring when the oxygenated media is introduced within the bioreactor using the return port.

In some implementations, the method further comprises cleaning the screen using flow of the mixture from the vertical wheel.

In some implementations, removing the portion of the first media through the output port of the bioreactor comprises using a peristaltic pump.

In some implementations, treating the second media within the media preparation container comprises using a membrane exchanger to produce the oxygenated media.

In some implementations, the membrane exchanger comprises a biocompatible membrane.

In some implementations, the bioreactor comprises a single-use bioreactor.

In some implementations, the bioreactor comprises a large-scale bioreactor.

In some implementations, the bioreactor comprises a vertical wheel.

In another implementation, the present disclosure provides a bioreactor system including a bioreactor to contain a mixture of at least cells, and a first media. The bioreactor includes an output port to remove a portion of the first media, a screen covering the output port to retain the cells within the bioreactor, and a return port. The media preparation container is external to the bioreactor and fluidly coupled to the return port, the media preparation container to receive a second media. The media treatment device is to treat the second media within the media preparation container to produce oxygenated media within a threshold pH range, wherein the system is configured to move the oxygenated media from the media preparation container to the bioreactor through the return port of the bioreactor.

In some implementations, the second media comprises the portion of the first media, and the media preparation container is fluidly coupled to the output port of the bioreactor for receiving the portion of the first media.

In some implementations, the second media is separate from the portion of the first media, and further comprising a waste container fluidly coupled to the output port of the bioreactor for receiving the portion of the first media.

In some implementations, the media treatment device comprises an external oxygenator.

In some implementations, the media treatment device comprises a gas exchange device. In some implementations, the media treatment device comprises an membrane exchanger.

In some implementations, the media treatment device comprises a sparger.

In some implementations, the media treatment device comprises a supply of at least one of oxygen, carbon dioxide, air, nutrients, or a liquid base.

In some implementations, the bioreactor system further comprises a sensor and a controller, wherein the sensor is to determine a parameter value within the mixture and the controller is to compare the determined parameter value to a reference parameter value, determine a difference between the determined parameter value and the reference parameter value being greater than a threshold value, and determine a volume of the portion of the first media to remove from the port of the bioreactor to allow a subsequently determined parameter value after introducing the oxygenated media within the bioreactor to be within a threshold of the reference parameter value.

In some implementations, the parameter value comprises a value of dissolved oxygen within the mixture.

In some implementations, the parameter value comprises a value of dissolved carbon dioxide within the mixture.

In some implementations, the parameter value comprises a pH value of the mixture.

In some implementations, the bioreactor system further comprises a pump to remove the portion of the first media through the output port of the bioreactor.

In some implementations, the bioreactor system further comprises a heater to warm the second media within the media preparation container.

In some implementations, the media treatment device comprises a membrane exchanger comprising a first chamber, a second chamber, and a membrane separating the first chamber and the second chamber.

In some implementations, the bioreactor system further comprises a vertical wheel to mix the mixture of the cells and the first media in the bioreactor.

In other implementations, the present disclosure provides a computer-readable memory coupled to one or more processors and storing instructions thereon that, when executed by the one or more processors, cause the one or more processors to perform a process on a bioreactor system. The process including operate a vertical wheel in a large-scale bioreactor to mix a mixture of at least cells and a first media in the large-scale bioreactor. The process further including actuating a pump to remove a portion of the first media through an output port of the bioreactor introduce a second media into a media preparation container external to the bioreactor, the cells being retained within the bioreactor using a screen covering the output port. The process further including operating a media treatment device to treat the second media within the media preparation container to produce oxygenated media within a threshold pH range. The process further including actuating a second pump to introduce the oxygenated media to the bioreactor using a return port of the bioreactor.

In other implementations, the present disclosure provides a method of using a large-scale bioreactor system. The method includes a) mixing a mixture of at least cells, and media using a vertical wheel in a single-use bioreactor. The method further includes b) flowing the mixture from the bioreactor and into a first chamber of a membrane exchanger at a first flow rate. The method further includes c) pumping oxygen into a second chamber of the membrane exchanger at a second flow rate, the second chamber separated from the first chamber by a membrane. The method further includes d) diffusing the oxygen in the second chamber through the membrane of the membrane exchanger and into the mixture within the first chamber to produce oxygenated media.

In some implementations, the method further comprises determining an oxygen value within the mixture in the bioreactor, comparing the determined oxygen value to a reference oxygen value, determining a difference between the determined oxygen value and the reference oxygen value being greater than a threshold, and determining the first flow rate and the second flow rate to allow a subsequently determined oxygen value of the mixture after introducing the oxygenated media to the bioreactor to be within a threshold of the reference oxygen value.

### BRIEF DESCRIPTION OF THE DRAWINGS

FIG. 1 is a schematic diagram of an example large-scale bioreactor system in accordance with the teachings of this disclosure.

FIG. 2 is a schematic diagram of an implementation of a media treatment device that can be used to implement the media treatment device of the present disclosure.

FIG. 3 is a flowchart of an example process of using the bioreactor system of FIG. 1 or any of the other disclosed implementations.

FIG. 4 is a schematic diagram of an alternative example large-scale bioreactor system in accordance with the teachings of this disclosure.

FIG. 5 is a flowchart of an example process of using the bioreactor system of FIG. 4 or any of the other disclosed implementations.

### DETAILED DESCRIPTION

Although the following description discloses a detailed description of implementations of methods, apparatuses, and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

The commercial potential of cell therapies requires overcoming the challenges related to scalable manufacturing of living cells. Therapeutic cells are typically human-derived, and as aerobic organisms they require constant management of metabolic nutrients and waste products. Furthermore, they are also typical anchorage-dependent and grow as cell aggregates or on the surface of plastic microcarriers while suspended in liquid medium. For cell culture processes performed in bioreactors, oxygen must be dissolved into the liquid medium while dissolved carbon dioxide (CO₂) produced by the cells must be removed.

A single dose of a therapeutic cell therapy for a single patient can require billions or trillions of cells. For allogeneic cell therapies that aim to service large patient populations, a robust and scalable manufacturing process in bioreactors is necessary to safely and efficiently produce vast numbers of cells. An example of target concentration of therapeutic cells at commercial scale is 1 to 1.5 million cells per milliliter of medium. At larger volume bioreactors (100 or 500+L), this equates to of trillions of viable cells in the bioreactor, with all of them consuming dissolved oxygen and producing CO₂.

At smaller volumetric scales, gas exchange through the gaseous headspace above the liquid level inside the bioreactor is typically sufficient to add and remove oxygen and CO₂, respectively. As the physical dimensions and volumetric scale of a bioreactor increases, the surface area of the liquid in contact with the headspace will likely increase much faster than the volume of the headspace itself, and thus headspace gas exchange alone may become insufficient. Furthermore, the density and metabolic demands of living cells exponentially increases as bioreactor volume increases, and thus the management of dissolved oxygen and CO₂ will become even more difficult at larger scales, such as those required for commercial production of allogeneic cell therapies.

Oxygen dissolved in the liquid medium is often the limiting factor for suspension-based cell culture processes. Cells will cease to grow in number if available dissolved oxygen is nearly or completely consumed, and if oxygen is not replaced the cells may even begin to die. In order to maximize concentration of cells in a bioreactor and thus the total process yield, sufficient levels of oxygen must be maintained in the liquid medium. For bacteria and yeast cultures, the critical oxygen concentration is usually 10-50% of air saturation as an example. For cultures of human-derived cells, the critical concentration is around 50% or even greater as an example.

CO₂ produced as metabolic waste by aerobic cells is another parameter that must be monitored during cell culture processes. The pH of the liquid medium will decrease as the amount of dissolved CO₂ increases. If dissolved CO₂ accumulates to sufficient levels, the pH may become acidic enough to inhibit growth or even damage suspended cells. Removing or stripping CO₂ from liquid medium can be accomplished by introducing bubbles of gas such as pure oxygen or air with a much lower concentration of CO₂ compared to what is dissolved in the medium. Stripping will naturally occur as CO₂ molecules will move out of the liquid medium and into the gas bubbles in an attempt to reach equilibrium.

The gas transfer rate (from a gaseous source into the liquid medium) depends on the difference of gas concentrations and the volumetric mass transfer coefficient kLa, which describes the efficiency of gas transport into liquid across the surface area of contact between the gas and liquid. The gas transfer rate of oxygen and CO₂ can be different based on various factors such as temperature, agitation conditions, and different kLa of the gasses.

In smaller-scale, stirred-type bioreactors, oxygen is pumped (as a mixture of gasses along with carbon dioxide and nitrogen) into the vessel's headspace, which can provide a large gas-liquid contact surface area (depending on vessel geometry). In addition, supplemental oxygen can also be sparged directly into the liquid medium through a controllable port located within the liquid medium. Oxygen gas bubbles, with average size dictated by the port opening's diameter, rise upwards through the liquid medium due to their buoyancy. Together, the headspace contact area and rising bubbles were also sufficient to strip CO₂ from the liquid medium and prevent a significant rise in pH.

Generally, k_{L}a increases with smaller bubbles, as they provide a larger gas-liquid interface per unit of liquid volume and spend longer in the medium compared to big bubbles, allowing more gas to be transferred into and out of the liquid. Medium density and composition, vessel geometry, and impeller agitation characteristics also affect gas transfer rate. Bioreactor design and process conditions must be able to provide an incoming oxygen transfer rate that is greater than the rate that cells can consume dissolved oxygen at peak demand, as well as removing dissolved CO₂ to prevent the liquid medium from becoming to acidic.

The volumetric mass transfer coefficient k_{L}a from headspace gassing in a bioreactor decreases at larger working volume as the gas-liquid interfacial area per liquid volume decreases. In traditional E. coli or CHO cell culture processes (e.g., for recombinant protein or monoclonal antibody production), a simple solution to meet the high oxygen uptake requirement is to sparge pure oxygen directly into the medium. However, a concern of small bubbles rising to the liquid surface layer is the formation of foam. Cells can become trapped on the surface of these foam bubbles and thus be removed from access to needed nutrients and agitation, ultimately resulting in a reduction of total cell yield during a cell culture process.

Anti-foaming chemical agents do exist that alleviate the formation of foam, but as a hydrophobic agent they can become incorporated into the membranes of cells. This was of minimal concern for traditional processes where proteins such as monoclonal antibodies were the desired product, and the cells were merely production hosts to be discarded. With cell therapies, the cells themselves are the product, and incorporated anti-foaming chemicals can present unknown effects on cells which poses a potential risk for human patients. The addition of anti-foaming agents or similar chemicals (with unknown risks to patients) are undesirable for cell therapy culture processes.

Additionally, bubbles may burst once they reach the surface layer of the liquid medium and cause hydrodynamic shear stress to cells, negatively affecting the viability of cells grown on microcarriers or as aggregates. In addition, depending on the weight of cells, the bursting action may throw the cells onto parts of the vessel above the liquid level, where they may attach and remain and thus reduce the total process yield. Reducing the average size of bubbles introduced into the medium may exacerbate foam formation.

Replacing a portion or most of the medium that has been depleted of oxygen (and other nutrients) is another method to provide oxygen to cells and also removing CO₂. As examples, step-wise removal of some volume of spent medium can be followed by addition of fresh medium. However, this process typically requires a pause in mixing, or waiting until numerous nutrients (not just oxygen) are depleted to certain levels; both can be detrimental to cell growth and quality. In a continuous perfusion method, medium is continuously removed from the bioreactor while fresh or replenished medium is re-added in a continuous cycle. Perfusion may require a specialized vessel feature, such as a filtered mesh screen, to retain cells without allowing them to clump and/or clog the port where medium is continuously removed. Furthermore, at larger volumetric scales, continuous addition and removal of specialized cell therapy medium can be cost-prohibitive.

A potential solution is to retain cells in the bioreactor while continuously removing O₂-depleted and CO₂-saturated medium to an external device or even second bioreactor. Oxygen enrichment of cell-free medium (up to 500% air saturation) can be done rapidly by sparging (using one or multiple sparging tubes) of highly concentrated or even pure O₂ without any worry of harming cells. Oxygen-rich medium may be quickly introduced back into original bioreactor to minimize time and maintain volume in the vessel. Traditional methods such as reducing liquid volume or increasing agitation rate in a given bioreactor system can also improve oxygen transfer rate, although the potential impact to overall cell yield and/or cell aggregate size and morphology will need to be assessed for each process.

As O₂ and CO₂ have different gas transfer rates and solubilities in liquid, and depending on factors such as duration and intensity of sparging, sufficient CO₂ stripping (indicated by a rise in pH to an acceptable range) may not be achieved simultaneously with desired super-saturation of O₂, especially at larger volumetric scales. In order to minimize time the liquid media spends in the external device and away from cells in the bioreactor, liquid base could be added to the external exchange device or bioreactor to counteract the lowering of pH due to CO₂ accumulation.

Another potential solution for enriching cell-containing medium without generating bubbles from sparging involves an external membrane exchanger. Membrane gas exchangers typically utilize diffusion principles, operating much like a shell-and-tube heat exchanger. Oxygen depleted medium from the bioreactor circulates through one side of the exchanger with oxygen being pumped into the other side. A highly permeable, biocompatible membrane, such as silicon, allows pure oxygen in the gas phase to diffuse into the medium as it circulates through the device. Setting the pump speed used to draw medium from the bioreactor and the flow rate of oxygen into the device will determine the k_{L}a. If the per cell oxygen consumption rate is known, operators can calculate the required circulation rate to maintain a sufficient oxygen concentration in the medium for cell survival and growth.

The disclosed implementations relate to systems and methods for replenishing dissolved oxygen consumed by therapeutic cells in a bioreactor during the course of a scalable cell culture process. Providing sufficient oxygen will optimize suspension-based cell culture processes involving cells grown on microcarriers or as aggregates, particularly at larger volumetric scales with correspondingly greater concentrations of cells. These systems and methods are applicable across a broad range of bioreactor sizes, from 0.1 L working volume for small-scale R&D use to 500 L working volume for large-scale clinical or commercial manufacturing.

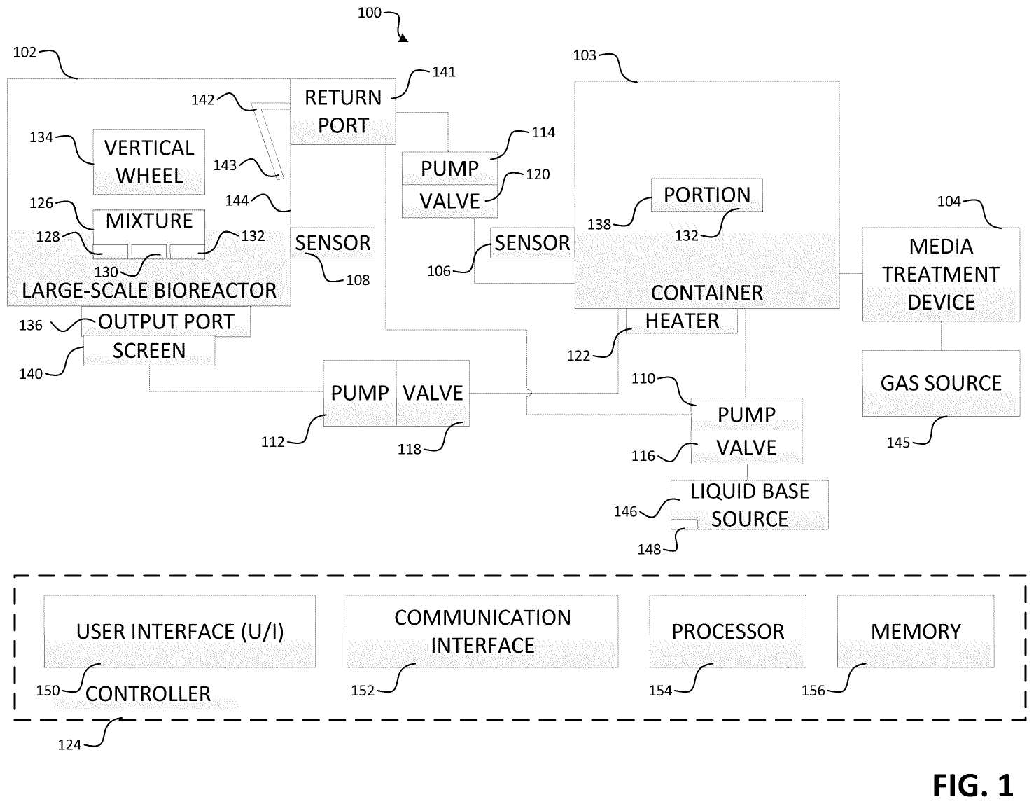

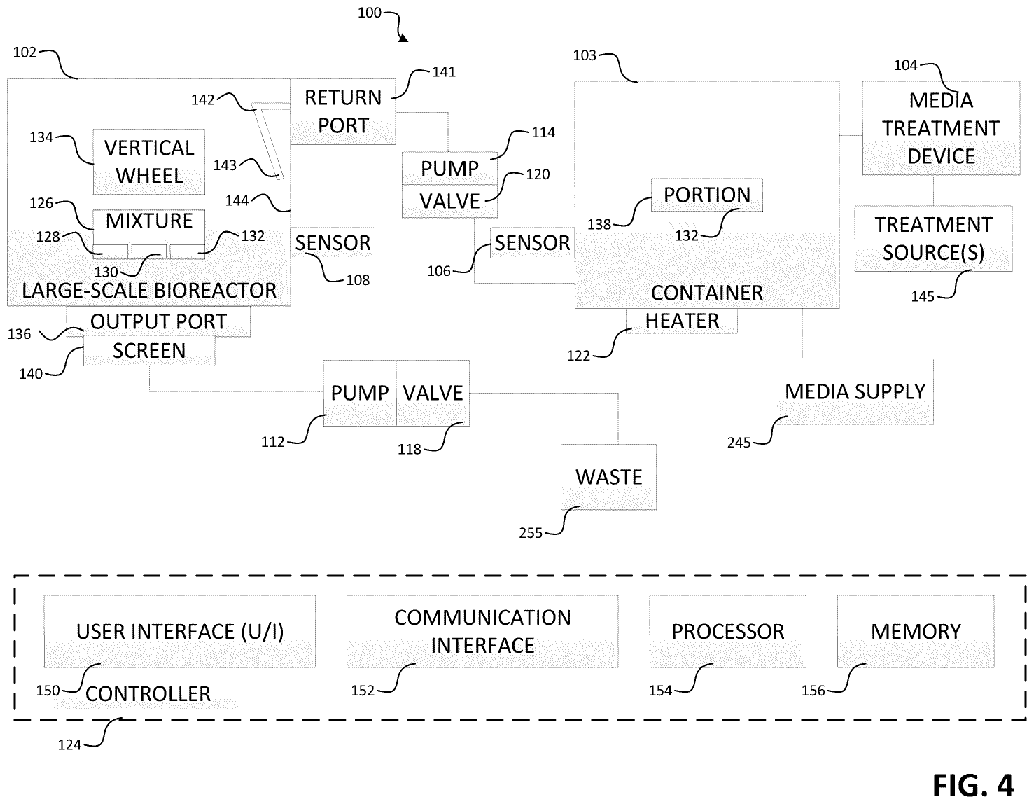

FIG. 1 is a schematic diagram of an example bioreactor system 100 in accordance with the teachings of this disclosure. FIG. 4, which will be described further below, is a schematic diagram of an alternative example bioreactor system 100 in accordance with the teachings of this disclosure.

In FIG. 1, the bioreactor system 100 includes a bioreactor 102 (which may be a large-scale bioreactor), a container 103 external to the bioreactor 102 (also referred to herein as a media preparation container), a media treatment device 104, and a plurality of sensors 106, 108. The bioreactor system 100 also includes a plurality of pumps 110, 112, 114, a plurality of valves 116, 118, 120, a heater 122, and a controller 124. The controller 124 is electrically and/or communicatively coupled to the bioreactor 102, the container 103, the media treatment device 104, the plurality of sensors 106, 108, the plurality of pumps 110, 112, 114, the plurality of valves 116, 118, 120, the heater 122 and is adapted to cause the bioreactor 102, the container 103, the media treatment device 104, the plurality of sensors 106, 108, the plurality of pumps 110, 112, 114, the plurality of valves 116, 118, 120, the heater 122 to perform various functions as disclosed herein. While the bioreactor system 100 is shown including the media treatment device 104, the plurality of sensors 106, 108, the plurality of pumps 110, 112, 114, the plurality of valves 116, 118, 120, and the heater 122, one or more of the media treatment device 104, the plurality of sensors 106, 108, the plurality of pumps 110, 112, 114, the plurality of valves 116, 118, 120, and the heater 122 may be omitted. Additionally, while the example of FIG. 1 depicts the plurality of pumps 110, 112, 114 and the plurality of valves 116, 118, 120 as being separate and connected to various other features of the system via fluidic lines, other examples of the disclosed system can combine any one or more of the pumps 110, 112, 114 and valves 116, 118, 120 into a common “pumping system”.

The bioreactor 102 contains a mixture 126 of optional microcarriers 128, cells 130, and media 132 in the implementation shown and has a vertical wheel 134 that mixes the mixture 126 of the optional microcarriers 128, the cells 130, and the media 132. The cells 130 may include pluripotent stem cells. The bioreactor 102 also includes an output port 136 that allows a portion 138 of the media 132 to be removed from the bioreactor 102 and a screen 140 that covers the output port 136 to retain the optional microcarriers 128 and the cells 130 within the bioreactor 102. The bioreactor 102 also includes a return port 141. The screen 140 may alternatively be in a different location or omitted. The screen 140 may be positioned between the container 103 and the media treatment device 104. A portion 138 of the mixture 126 may, thus, flow through the output port 136 and into the container 103 and the screen 140 positioned between the container 103 and the media treatment device 104 may retain the optional microcarriers 128 and the cells 130 within the container 103 and allow the media 132 to flow into the media treatment device 104, for example.

The container 103 is fluidly coupled to the output port 136 and the return port 141 and receives the portion 138 of the media 132 from the output port 136 in operation. The media treatment device 104 treats the portion 138 of the media 132 within the container 103 to produce oxygenated media within a threshold pH range. The oxygenated media is introduced to the bioreactor 102 using the return port 141 of the bioreactor 102. The return port 141 can include a tube 142 having a distal end 143 that tapers toward an internal surface 144 of the bioreactor 102 to prevent or inhibit splashing from occurring when the oxygenated media is introduced to the bioreactor 102 using the return port 141.

The media 132 may be treated by oxygenating the portion 138 of the media 132 and/or by removing dissolved carbon dioxide from the portion 138 of the media 132. The media treatment device 104 can, thus, be an external oxygenator, a gas exchange device, and/or a membrane exchanger. The bioreactor system 100 also includes a gas source 145 in the implementation shown that can provide gas such as oxygen to the media treatment device 104.

The sensor 108 determines a parameter value within the mixture 126 and the controller 124 compares the determined parameter value to a reference parameter value. The controller 124 may compare the values to determine if a difference between the determined parameter value and the reference parameter value is greater than a threshold value. The controller 124 determines a volume of the portion 138 of the media 132 to remove from the output port 136 of the bioreactor 102 if the difference is greater than the threshold value to allow a subsequently determined parameter value after introducing the oxygenated media within the bioreactor 102 to be within a threshold of the reference parameter value. The bioreactor system 100 thus treats a volume of the media 132 and introduces that oxygenated media into the bioreactor 102 to enable the parameter values within the bioreactor 102 to satisfy a threshold value. The parameter value may be a value of dissolved oxygen within the mixture 126, a value of dissolved carbon dioxide within the mixture 126, and/or a pH value of the mixture 126. The parameter value determined by the sensor 108, however, may be any other parameter.

The sensor 106 may similarly measure a parameter value of the portion 138 of the mixture 132 within the container 103 and the controller 124 can determine when the parameter value measured is within a threshold of a reference parameter value. The controller 103 may determine when an oxygen content value of the portion 138 of the media 132 is within a threshold of a reference oxygen content value and thereafter cause the oxygenated media to be introduced within the bioreactor 102, for example. The controller 103 may determine when a pH value of the portion 138 of the media 132 is within a threshold of a reference pH value and thereafter cause the oxygenated media to be introduced within the bioreactor 102 as an alternative for example.

The pump 112 may be used to remove the portion 138 of the media 132 through the output port 136 of the bioreactor 102 and the pump 114 may be used to introduce the oxygenated media 132 into the bioreactor 102. The pumps 112, 114 may be peristaltic pumps. Other types of pumps may prove suitable and/or one or more of the pumps 112, 114 may be omitted. The heater 122 is positioned to warm the portion 138 of the media 132.

The bioreactor system also includes a liquid base source 146 in the implementation shown containing a liquid base 148. The liquid base source 146 is fluidly coupled to the container 103 and the bioreactor 102. The sensor 106 determines a parameter value of the mixture 126 and/or the sensor 108 measures a parameter value of the portion 138 of the media 132 in operation and the controller 124 determines if the determined parameter values are within a threshold of reference parameter values. The controller 124 causes the pump 110 and/or the valve 116 to actuate to flow the liquid base 148 to the container 103 and/or to the bioreactor 102 if the measured parameter values are outside of the threshold of the reference parameter values, for example.

The controller 124 includes a user interface 150, a communication interface 152, one or more processors 154, and a memory 156 storing instructions executable by the one or more processors 154 to perform various functions including the disclosed implementations. The user interface 150, the communication interface 152, and the memory 156 are electrically and/or communicatively coupled to the one or more processors 154.

The user interface 150 is adapted to receive input from a user and to provide information to the user associated with the operation of the bioreactor system 100 and/or a process taking place in an implementation. The user interface 150 may include a touch screen, a display, a key board, a speaker(s), a mouse, a track ball, and/or a voice recognition system. The touch screen and/or the display may display a graphical user interface (GUI).

The communication interface 152 is adapted to enable communication between the bioreactor system 100 and a remote system(s) (e.g., computers) via a network(s) in an implementation. The network(s) may include the Internet, an intranet, a local-area network (LAN), a wide-area network (WAN), a coaxial-cable network, a wireless network, a wired network, a satellite network, a digital subscriber line (DSL) network, a cellular network, a Bluetooth connection, a near field communication (NFC) connection, etc. Some of the communications provided to the remote system may be associated with analysis results, imaging data, etc., generated or otherwise obtained by the bioreactor system 100. Some of the communications provided to the bioreactor system 100 may be associated with therapeutic cell growth processes and/or a protocol(s) to be executed by the bioreactor system 100.

The one or more processors 154 and/or the bioreactor system 100 may include one or more of a processor-based system(s) or a microprocessor-based system(s). The one or more processors 154 and/or the bioreactor system 100 in some implementations includes one or more of a programmable processor, a programmable controller, a microprocessor, a microcontroller, a graphics processing unit (GPU), a digital signal processor (DSP), a reduced-instruction set computer (RISC), an application specific integrated circuit (ASIC), a field programmable gate array (FPGA), a field programmable logic device (FPLD), a logic circuit, and/or another logic-based device executing various functions including the ones described herein.

The memory 156 can include one or more of a semiconductor memory, a magnetically readable memory, an optical memory, a hard disk drive (HDD), an optical storage drive, a solid-state storage device, a solid-state drive (SSD), a flash memory, a read-only memory (ROM), erasable programmable read-only memory (EPROM), electrically erasable programmable read-only memory (EEPROM), a random-access memory (RAM), a non-volatile RAM (NVRAM) memory, a compact disc (CD), a compact disc read-only memory (CD-ROM), a digital versatile disk (DVD), a Blu-ray disk, a redundant array of independent disks (RAID) system, a cache and/or any other storage device or storage disk in which information is stored for any duration (e.g., permanently, temporarily, for extended periods of time, for buffering, for caching).

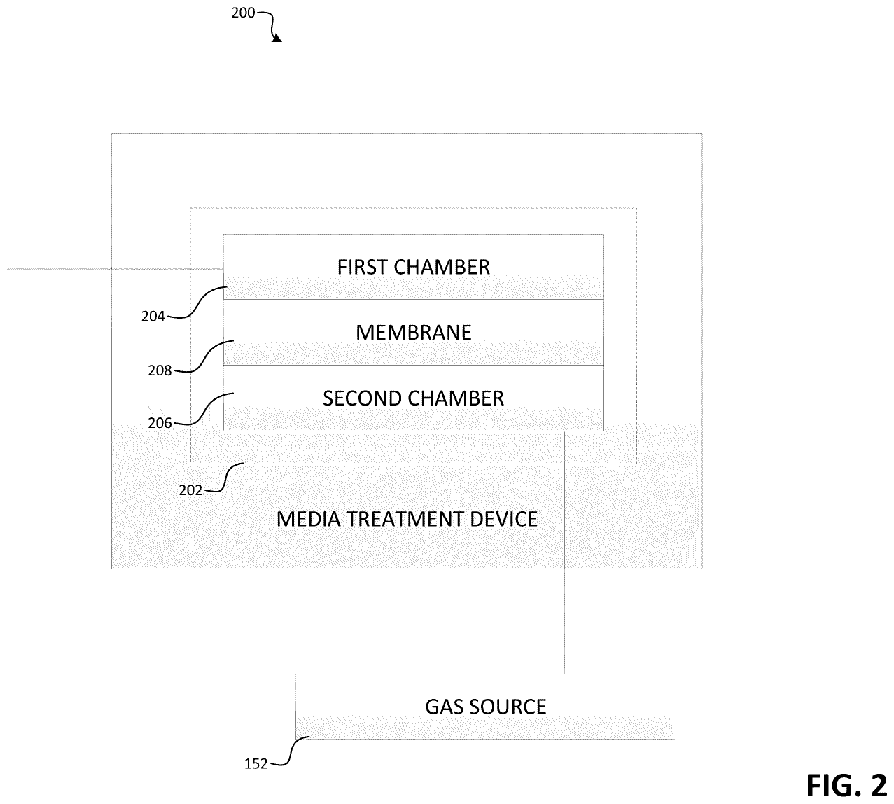

FIG. 2 is a schematic diagram of an implementation of a media treatment device 200 that can be used to implement the media treatment device 104 of FIG. 1. The media treatment device 200 of FIG. 2 is a membrane exchanger 202 having a first chamber 204, a second chamber 206, and a membrane 208 separating the first chamber 204 and the second chamber 206. The membrane 208 may be a biocompatible membrane.

The portion 138 of the media 132 is flowed into the first chamber 204 in operation at a flow rate and oxygen is pumped into the second chamber 206 at a flow rate. The oxygen diffuses through the membrane 208 and into the portion 138 of the media 132 within the first chamber 204 to produce oxygenated media. The sensor 106 can be used to measure an oxygen value within the portion 138. The controller 124 can compare the determined oxygen value to a reference oxygen value and determine a difference between the determined oxygen value and the reference oxygen value being greater than a threshold. The controller 124 can also determine the flow rate of flowing the portion 138 into the first chamber 204 and the flow rate of the oxygen into the second chamber 206 to allow a subsequently determined oxygen value of the mixture after introducing the oxygenated media within the bioreactor to be within a threshold of the reference oxygen value. Put another way, the flow rates into the media treatment device 200 may be dynamically adjusted to allow the measured values to be within a threshold range of the reference values. The mixture 126 including the optional microcarriers 128, the cells 130, and/or the media 132 can alternatively be flowed into the first chamber 204.

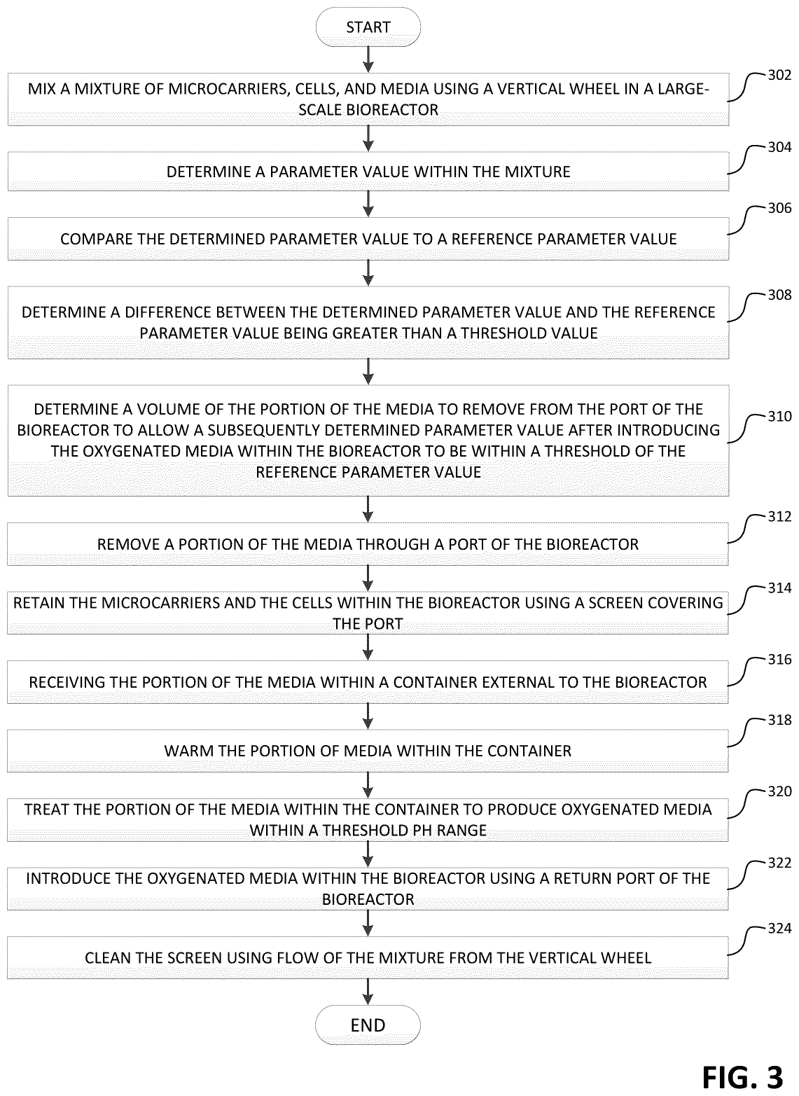

FIG. 3 is a flowchart of an example process 300 of using the bioreactor system 100 of FIG. 1 or any of the other disclosed implementations. The order of execution of the blocks may be changed, and/or some of the blocks described may be changed, eliminated, combined, and/or subdivided into multiple blocks.

The process of 300 of FIG. 3 begins with the mixture 126 of the optional microcarriers 128, the cells 130, and the media 132 being mixed using the vertical wheel 134 in the large-scale bioreactor 102 (Block 302). The cells 130 may include pluripotent stem cells.

A parameter value within the mixture 126 is determined (Block 304). The sensor 108 can be used to determine the parameter value. The determined parameter value is compared to a reference parameter value (Block 306). The controller 124 can compare the determined parameter value and the reference parameter value and the reference parameter may be stored in the memory 156, for example. A difference between the determined parameter value and the reference parameter value is determined to be greater than a threshold value (Block 308) and a volume of the portion 138 of the media 132 to remove from the output port 136 of the bioreactor 102 to allow a subsequently determined parameter value after introducing the oxygenated media within the bioreactor 102 to be within a threshold of the reference parameter value is determined (Block 310). The controller 124 can be used to determine the difference between the parameter value and the reference parameter value and/or the volume of the portion 138 of the media 132 to remove from the output port 136 of the bioreactor 102. The parameter value may include a value of dissolved oxygen within the mixture 126, a value of dissolved carbon dioxide within the mixture 126, and/or a pH value of the mixture 126.

The portion 138 of the media 132 is removed through the output port 136 of the bioreactor 102 (Block 312). The portion 138 of the media 132 can be removed through the output port 136 of the bioreactor 102 using the pump 112 that may be implemented by a peristaltic pump. Other ways of removing the portion 138 may prove suitable such as gravity, for example.

The optional microcarriers 128 and the cells 130 are retained within the bioreactor 102 using the screen 140 covering the output port 136 (Block 314) and the portion 138 of the media 132 is received within the container 103 external to the bioreactor 102 (Block 316). The portion 138 of the media 132 may be warmed (Block 318). The heater 122 may be used to warm (e.g., heat) the portion 138 of the media.

The portion 138 of the media 132 is treated within the container 103 to produce oxygenated media within a threshold pH range (Block 320). The sensor 106 may be used to determine the O₂ and/or pH value of the portion 138 and the controller 124 may be used to determine if the determined pH value is within the threshold pH range. The sensor 106 can in some exampled includes multiple sensors for measuring O₂, pH, nutrients, and/or other parameters. The portion 138 of the media 132 may be treated by oxygenating the portion 138 of the media 132, removing dissolved carbon dioxide from the media 132, and/or adding the liquid base 148 to the container 103. The portion 138 of the media 132 may also or alternatively be treated using an external gas exchange device, a gas exchanger, and/or a membrane exchanger having a biocompatible membrane. The portion 138 of the media 132 may also or alternatively include sparging the portion 138 within the container 103. The sparging may occur without the use of surfactants and without the use of anti-foaming agents.

The oxygenated media is introduced within the bioreactor 102 using the return port 141 of the bioreactor 102 (Block 322). The oxygenated media can be introduced within the bioreactor 102 by introducing the oxygenated media within the bioreactor 102 using the return port 141 having the tube 142 having the distal end 143 that tapers toward the internal surface 144 of the bioreactor 102. Splashing within the bioreactor 102 is prevented or inhibited from occurring as a result when the oxygenated media is introduced within the bioreactor 102 using the return port 141. The processes of blocks 312, 314, 316, 320, and 322 can be occur substantially continually in some implementations. The process of block 302 may stop while the processes of blocks 312, 314, 316, 320, and 322 occur in some implementations. The processes of blocks 312, 314, 316, 320, and 322 may occur on demand. The screen 140 is cleaned using flow of the mixture from the vertical wheel 134 (Block 324).

As mentioned above, FIG. 4 depicts an alternative example of a bioreactor system 100 in accordance with the teachings of the present disclosure. The system 100 of FIG. 4 is similar to FIG. 1 in many respects and, as such, only the key distinctions will be described in detail. Otherwise, the description set forth above related to aspects of FIG. 1 that are common to FIG. 4 equally apply to FIG. 4. The primary distinction between the example of FIG. 1 and the alternative of FIG. 4 is that the system of FIG. 4 is configured to move the portion of the spent media withdrawn from the output port 136 of the bioreactor 102 to a waste container 255 as opposed to the media preparation container 103 of FIG. 1. As such, the media ultimately provided to the media preparation container 103 is separate from the media withdrawn from the bioreactor 103. In the version depicted in FIG. 4, the media provided to the media preparation container 103 is supplied from a media supply 245, which may include a pump or other mechanism to facilitate transfer of the media from a source container, for example, to the media preparation container 103. In other versions, the media could be supplied to the media preparation container 103 by other means including simply pouring the media into the container 103. This alternative example in FIG. 4 may be desirable in applications where the spent media withdrawn from bioreactor will require a significant treatment including the addition of O₂ and nutrients, for example, as well as the removal of CO₂ and waste products. Depending on the amount of processing required for the spent media in the example of FIG. 1, for example, it may be preferred in certain circumstances/applications to instead direct the spent media to waste and introduce fresh media into media preparation container because the fresh media may require less processing (e.g., preconditioning with some level of dissolved O₂, pH, CO₂, nutrients, etc.).

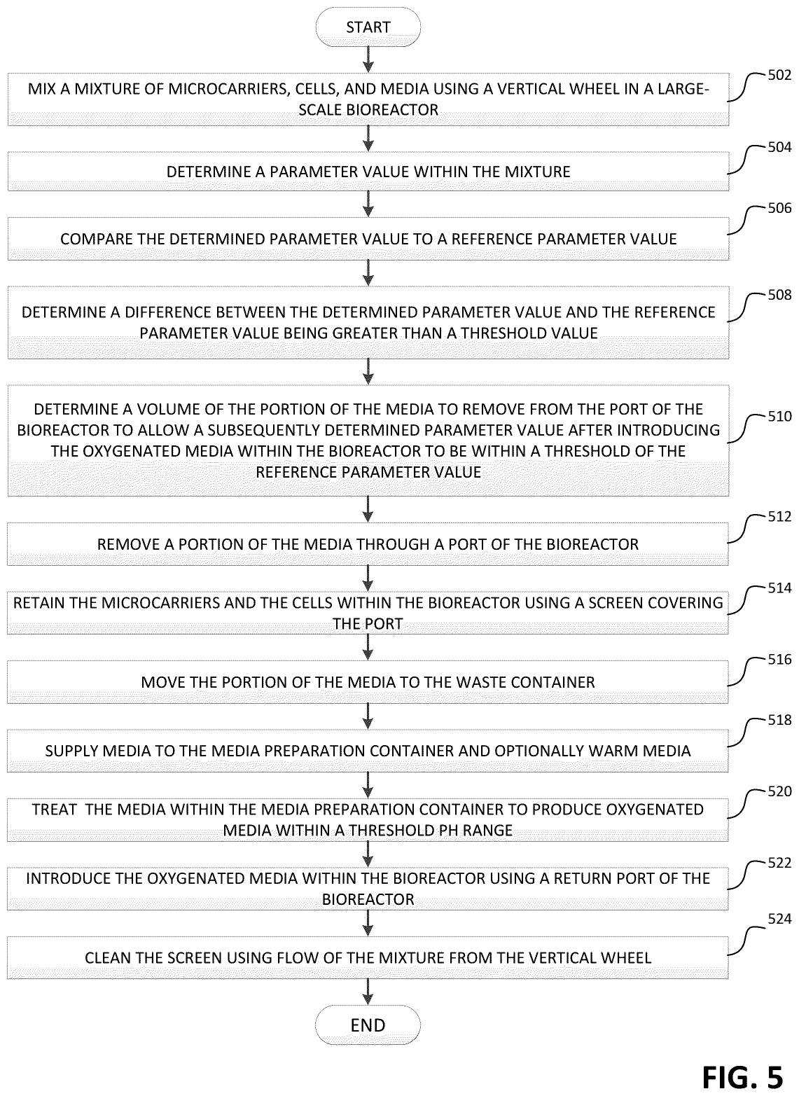

Based on the distinctions above, FIG. 5 is a flowchart of an example process 500 of using the bioreactor system 100 of FIG. 4 or any of the other disclosed implementations. The order of execution of the blocks may be changed, and/or some of the blocks described may be changed, eliminated, combined, and/or subdivided into multiple blocks.

The process of 500 of FIG. 5 begins with the mixture 126 of the optional microcarriers 128, the cells 130, and the media 132 being mixed using the vertical wheel 134 in the large-scale bioreactor 102 (Block 502). The cells 130 may include pluripotent stem cells.

A parameter value within the mixture 126 is determined (Block 504). The sensor 108 can be used to determine the parameter value. The determined parameter value is compared to a reference parameter value (Block 506). The controller 124 can compare the determined parameter value and the reference parameter value and the reference parameter may be stored in the memory 156, for example. A difference between the determined parameter value and the reference parameter value is determined to be greater than a threshold value (Block 508) and a volume of the portion 138 of the media 132 to remove from the output port 136 of the bioreactor 102 to allow a subsequently determined parameter value after introducing the oxygenated media within the bioreactor 102 to be within a threshold of the reference parameter value is determined (Block 510). The controller 124 can be used to determine the difference between the parameter value and the reference parameter value and/or the volume of the portion 138 of the media 132 to remove from the output port 136 of the bioreactor 102. The parameter value may include a value of dissolved oxygen within the mixture 126, a value of dissolved carbon dioxide within the mixture 126, and/or a pH value of the mixture 126.

The portion 138 of the media 132 is removed through the output port 136 of the bioreactor 102 (Block 512). The portion 138 of the media 132 can be removed through the output port 136 of the bioreactor 102 using the pump 112 that may be implemented by a peristaltic pump. Other ways of removing the portion 138 may prove suitable such as gravity, for example.

The optional microcarriers 128 and the cells 130 are retained within the bioreactor 102 using the screen 140 covering the output port 136 (Block 514) and the portion 138 of the media 132 is moved to the waste container 255 (Block 516).

Separately, media is provided to the container 103 from the media supply 245 and may optionally be warmed (Block 518). The heater 122 may be used to warm (e.g., heat) the media in the media preparation container 103.

The media 132 is treated within the container 103 to produce oxygenated media within a threshold pH range (Block 520). The sensor 106 may be used to determine the O₂, CO₂, nutrients, and/or pH value of the media and the controller 124 may be used to determine if the determined pH value is within the threshold pH range. The sensor 106 can in some exampled includes multiple sensors for measuring O₂, pH, CO₂, nutrients, and/or other parameters. The media 132 may be treated by oxygenating the portion 138 of the media 132, removing dissolved carbon dioxide from the media 132, and/or adding the liquid base 148 to the container 103. This treatment may include adding O₂, CO₂, nutrients, a liquid base, nutrients, or other components, or may include stripping dissolved CO₂. Adding components may include adding components by way of introduction into the head space of the container 103 and/or by sparging. The sparging may occur without the use of surfactants and without the use of anti-foaming agents. Moreover, treatment may include agitating the media in the container 103 with a mixing device such as a spinner mixer or vertical wheel mixer. The media may also or alternatively be treated using an external gas exchange device, a gas exchanger, and/or a membrane exchanger having a biocompatible membrane, such as those described above with respect to the example of FIGS. 1-3.

The oxygenated media is introduced within the bioreactor 102 using the return port 141 of the bioreactor 102 (Block 522). The oxygenated media can be introduced within the bioreactor 102 by introducing the oxygenated media within the bioreactor 102 using the return port 141 having the tube 142 having the distal end 143 that tapers toward the internal surface 144 of the bioreactor 102. Splashing within the bioreactor 102 is prevented or inhibited from occurring as a result when the oxygenated media is introduced within the bioreactor 102 using the return port 141. The process steps of blocks 512, 514, 516, 520, and 522 can be occur substantially continually in some implementations. The process of block 502 may stop while the process steps of blocks 512, 514, 516, 520, and 522 occur in some implementations. The process steps of blocks 512, 314, 516, 520, and 522 may occur on demand. The screen 140 is cleaned using flow of the mixture from the vertical wheel 134 (Block 524).

While FIGS. 1-3 and FIGS. 4-5 have been disclosed herein as different examples, the scope of the disclosure includes examples where certain or all features of FIGS. 1-3 and FIGS. 4-5 can be combined. For example, the present disclosure describes FIG. 1 as moving spent media from the bioreactor 102 to the external container 103 for treatment and return to the bioreactor 102. Alternatively, the disclosure describes the example of FIG. 4 as moving the spent media to waste while providing the media preparation container 103 with separate media (e.g., fresh media) for treatment (e.g., pre-treatment) and provision to the bioreactor 102. In some examples, a system in accordance with the present disclosure may combine these examples such that at least some of the portion of spent media withdrawn from the bioreactor is provided to the media preparation container 103 and mixed with the separate media provided by the media supply 245 of FIG. 4. In other examples, the controller may analyze the parameters of the portion of the spent media withdrawn from the bioreactor 102 and make a determination as whether to move some or all of that spent media to waste or to the media preparation container 103.

As discussed herein, the systems and methods of the present disclosure provide continual replenishment of O₂ to the mixture in the bioreactor while removing dissolved CO₂ to maintain desired pH and ensure a healthy environment for production of living cells at various cell culture process scales. Advantageously, the disclosed systems and methods achieve this by balancing the outflow of O₂ depleted media with the inflow of replenishing super-oxygenated media. In some examples, this balance can be characterized by the following mass balance equation applied to the dynamic consumption of cells in the bioreactor:

$$dQ / dt = [ {V}_{reactor} \times ( {dC}_{O 2} / dt ) ] / {C}_{O 2 in perfusion vessel} , where$$

$$dQ / dt : flow of super - oxygenated media into the bioreactor ( L / min )$$

$${V}_{reactor} : volume of media in bioreactor ( L )$$

$${dC}_{O 2} / dt : change in DO concentration \\ over time ( consumption ) in bioreactor [ ( g / L ) / min ]$$

$${C}_{O 2} in perfusion vessel : concentration \\ DO in perfusion ( external ) vessel ( g / L )$$

$$Numerator [ {V}_{reactor} \times ( {dC}_{O 2} / dt ) ] : total mass of \\ oxygen consumed in bioreactor ( g )$$

Each media component (i.e., dissolved O² (DO) and dissolved CO₂ (DCO₂)) has its own mass balance equation. However, for exponentially increasing cell population during an expansion process, DO is considered the more limiting factor. DO consumption typically outpaces DCO₂ production for human cells and, thus, a lack of DO will inhibit growth or damage cells before decreasing pH from CO₂ production can do the same.

A specific level or setpoint of DO in the bioreactor is targeted to maintain healthy cell expansion, whereas a specific target of CO₂ is less critical (broader range of pH is acceptable for human cells). Consumption needs to be calculated based on factors such as cell type, population doubling time, availability of other nutrients, etc.

Mass of O₂ in the bioreactor needs to be balanced between consumption by the cells and input of oxygen within the highly oxygenated media. The flow rate of media from external container can change depending on speed of consumption (exponentially increases with expanding cell population), concentration of oxygen in added media, and flow rate of spent media out of the bioreactor. For example, if flow rates in and out of the bioreactor are equal and unchanging, then the mass of O₂ enriched media being supplied to the reactor should be increased in accordance with demands of increasing cell population. The media used for cell cultures primarily consists of water, which has similar solubility behavior as that of gases (e.g., affected by temperature, atmospheric pressure, etc.)

It is understood that the foregoing examples are considered illustrative only of the principles of the invention. Further, since numerous modifications and changes will readily occur to those skilled in the art, it is not desired to limit the invention to the exact construction and operation shown and, accordingly, all suitable modifications and equivalents may be resorted to, falling within the scope of the invention.

What is claimed is:

1. A method of using a large-scale bioreactor system, comprising:
a) mixing a mixture of at least cells and a first media in a bioreactor;
b) removing a portion of the first media through an output port of the bioreactor;
c) retaining the cells within the bioreactor using a screen covering the output port;
d) receiving a second media within a media preparation container external to the bioreactor;
e) treating the second media within the media preparation container to produce oxygenated media within a threshold pH range; and
f) introducing the oxygenated media to the bioreactor using a return port of the bioreactor.

2. The method of claim 1, wherein the second media comprises the portion of the first media, and receiving the second media within the media preparation container comprises receiving the portion of the first media removed from the output port of the bioreactor.

3. The method of claim 1, further comprising moving the portion of the first media to a waste container after removing the portion of the first media from the bioreactor, and separately supplying the second media to the media preparation container.

4. The method of claim 1, wherein treating the second media comprises adding at least one of oxygen, air, carbon dioxide, or a liquid base to the second media.

5. The method of claim 1, wherein treating the second media comprises removing dissolved carbon dioxide from the second media.

6. The method of claim 1, wherein the cells comprise pluripotent stem cells and the mixture optionally includes microcarriers.

7. The method of claim 1, wherein treating the second media comprises treating the second media using an external gas exchange device.

8. The method of claim 1, wherein steps (b)-(f) occur substantially continuously and/or on demand.

9. The method of claim 1, wherein step (a) stops while steps (b)-(f) occur.

10. (canceled)

11. The method of claim 1, further comprising warming the second media within the media preparation container.

12. The method of claim 1, wherein treating the second media comprises sparging the second media within the media preparation container, wherein sparging is optionally performed without the use of surfactants and without the use of anti-foaming agents.

13. (canceled)

14. The method of claim 1, further comprising determining an oxygen value within the mixture, comparing the determined oxygen value to a reference oxygen value, determining a difference between the determined oxygen value and the reference oxygen value being greater than a threshold value, and determining a volume of the portion of the first media to remove from the output port of the bioreactor to allow a subsequently determined oxygen value after introducing the oxygenated media to the bioreactor to be within a threshold of the reference oxygen value.

15. The method of claim 1, further comprising determining a parameter value within the mixture, comparing the determined parameter value to a reference parameter value, determining a difference between the determined parameter value and the reference parameter value being greater than a threshold value, and determining a volume of the portion of the first media to remove from the output port of the bioreactor to allow a subsequently determined parameter value after introducing the oxygenated media to the bioreactor to be within a threshold of the reference parameter value.

16. The method of claim 15, wherein the parameter value comprises a value of dissolved oxygen within the mixture, a value of dissolved carbon dioxide within the mixture, or a pH value of the mixture.

17-21. (canceled)

22. The method of claim 1, wherein treating the second media within the media preparation container comprises using a membrane exchanger to produce the oxygenated media, wherein the membrane exchanger optionally comprises a biocompatible membrane.

23. (canceled)

24. The method of claim 1, wherein the bioreactor comprises a single-use bioreactor, a large-scale bioreactor, and/or a vertical wheel.

25-26. (canceled)

27. A bioreactor system, comprising:
a bioreactor to contain a mixture of at least cells and a first media and comprising:
an output port to remove a portion of the first media;
a screen covering the output port to retain the cells within the bioreactor; and
a return port;
a media preparation container external to the bioreactor and fluidly coupled to the return port, the media preparation container to receive a second media; and
a media treatment device to treat the second media within the media preparation container to produce oxygenated media within a threshold pH range, wherein the system is configured to move the oxygenated media from the media preparation container to the bioreactor through the return port of the bioreactor.

28. The bioreactor system of claim 27, wherein the second media comprises the portion of the first media, and the media preparation container is fluidly coupled to the output port of the bioreactor for receiving the portion of the first media.

29. The bioreactor system of claim 27, wherein the second media is separate from the portion of the first media, and further comprising a waste container fluidly coupled to the output port of the bioreactor for receiving the portion of the first media.

30. The bioreactor system of claim 27, wherein the media treatment device comprises an external oxygenator, a gas exchange device, a membrane exchanger, and/or a sparger.

31-33. (canceled)

34. The bioreactor system of claim 27, wherein the media treatment device comprises a supply of at least one of oxygen, carbon dioxide, air, nutrients, or a liquid base.

35. The bioreactor system of claim 27, further comprising a sensor and a controller, wherein the sensor is to determine a parameter value within the mixture and the controller is to compare the determined parameter value to a reference parameter value, determine a difference between the determined parameter value and the reference parameter value being greater than a threshold value, and determine a volume of the portion of the first media to remove from the port of the bioreactor to allow a subsequently determined parameter value after introducing the oxygenated media within the bioreactor to be within a threshold of the reference parameter value.

36. The bioreactor system of claim 35, wherein the parameter value comprises a value of dissolved oxygen within the mixture, a value of dissolved carbon dioxide within the mixture, or a pH value of the mixture.

37-38. (canceled)

39. The bioreactor system of claim 27, further comprising a pump to remove the portion of the first media through the output port of the bioreactor and/or a heater to warm the second media within the media preparation container.

40. (canceled)

41. The bioreactor system of claim 27, wherein the media treatment device comprises a membrane exchanger comprising a first chamber, a second chamber, and a membrane separating the first chamber and the second chamber.

42. The bioreactor system of claim 27, further comprising a vertical wheel to mix the mixture of the cells and the first media in the bioreactor.

43. (canceled)

44. A method of using a large-scale bioreactor system, comprising:
a) mixing a mixture of at least cells and media using a vertical wheel in a single-use bioreactor;
b) flowing the mixture from the bioreactor and into a first chamber of a membrane exchanger at a first flow rate;
c) pumping oxygen into a second chamber of the membrane exchanger at a second flow rate, the second chamber separated from the first chamber by a membrane; and
d) diffusing the oxygen in the second chamber through the membrane of the membrane exchanger and into the mixture within the first chamber to produce oxygenated media.

45. The method of claim 44, further comprising determining an oxygen value within the mixture in the bioreactor, comparing the determined oxygen value to a reference oxygen value, determining a difference between the determined oxygen value and the reference oxygen value being greater than a threshold, and determining the first flow rate and the second flow rate to allow a subsequently determined oxygen value of the mixture after introducing the oxygenated media to the bioreactor to be within a threshold of the reference oxygen value.
